(19) United States
(12) Patent Application Publication
(10) Pub. No.: US 2004/0131686 A1
(43) Pub. Date: Jul. 8, 2004

(54) Composition and method for treatment of bacterial vaginal infections

(76) Inventors: Robert J. Borgman, Mundelein, IL (US); James E. Juul, Wauconda, IL (US)

Correspondence Address: OLSON & HIERL, LTD., 36th Floor, 20 North Wacker Drive, Chicago, IL, 60606, US

(21) Appl. No.: 10/337,005

(22) Filed: Jan. 6, 2003

(51) Int. Cl. A61K 9/14; A61K 31/135

(52) U.S. Cl. 424/486; 514/649

(57) ABSTRACT

A gelled pharmaceutical composition suitable for the treatment of a bacterial vaginal infection comprises, on a total composition weight basis, about 0.1 to about 2 weight percent of benzydamine hydrochloride, about 0.5 to about 4 weight percent of a carbomer polymer, and about 3 to about 50 weight percent of propylene glycol, in water having a pH in the range of about 3.5 to about 6. Optionally, the gelled compositions can also contain polyethylene glycols, physiologically tolerable preservatives and electrolytes, as well as other pharmaceutically acceptable excipients.

### FIELD OF THE INVENTION

The invention relates generally to compositions and treatments for vaginal infections. More particularly, the invention relates to compositions comprising carbomer polymers and benzydamine suitable for treatment of bacterial vaginal infections.

### BACKGROUND OF THE INVENTION

Bacterial vaginosis (BV) is associated with an increased volume of vaginal discharge having a foul, fishy odor. Vaginal pH is elevated from the normal range (pH 3-4) to values greater than or equal to about pH 4.7. The odor and elevated pH are caused by a high level of amines, most notably trimethylamine, in the vagina. These amines are volatilized when the pH is raised, for example, as with addition of KOH or interaction with semen. The vaginal discharge is homogenous in appearance as opposed to the flocculent discharge seen in candidiasis. In contrast to candidiasis and trichomoniasis, itching generally is not associated with BV. A microscopic examination of a wet mount of the vaginal discharge in BV reveals an absence of polymorphonuclear leukocytes (PMNs). In contrast, the presence of many PMNs in a vaginal discharge is indicative of trichomoniasis, gonorrhea, or chlamydial cervicitis.

Typically, a clinical diagnosis of BV is made if three or more of the following four clinical criteria are present: (1) a homogenous discharge; (2) a pH. of greater than or equal to about 4.7; (3) a “fishy” amine odor upon the addition of 10% KOH to discharge; (4) presence of epithelial clue cells representing greater than or equal to about 20% of vaginal epithelial cells.

U.S. Pat. No. 5,536,743 to Borgman describes a pH buffered, aqueous gel formulation of the antibiotic metronidazole for treatment of BV. While providing an effective treatment for BV, metronidazole gel has been reported to have some undesirable side effects in some patients, such as yeast vaginitis following therapy, vulvovaginal irritation, and gastrointestinal discomfort. In addition, metronidazole can have adverse interactions with alcohol ingested by the patient.

Benzydamine hydrochloride (B-HCl) is a non-steroidal anti-inflammatory drug (NSAID) that is commercially available in Europe and other countries for topical application. B-HCl has the dual advantage of being an analgesic as well as having anti-microbial activity. A cream formulation of B-HCl, available under the trade name TANTUM ROSA® from Angelini Pharmaceuticals, Rome, Italy, has been utilized in a number of countries as a topical treatment for vaginal infections. The cream formulation, however, has a disadvantage of being difficult to administer intravaginally, remains in contact with the vaginal tissue for a relatively short period of time, and provides relatively rapid delivery of the active agent (B-HCl). These factors lead to a necessity for multiple applications of the cream formulation over a six to ten day period in order to treat a vaginal infection.

A desirable treatment for vaginal infections would be a composition for intravaginal administration that delivers the active agent over an extended period of time and remains in contact with the vaginal tissue for a time period sufficient for substantially all of the active agent to be released. The present invention provides such a desirable treatment in the form of an aqueous carbomer-based gel formulation of B-HCl.

### SUMMARY OF THE INVENTION

A gelled pharmaceutical composition useful for treating vaginal infections comprises, on a total composition weight basis, about 0.1 to about 2 weight percent of benzydamine hydrochloride, about 1 to about 4 weight percent of a carbomer polymer, and about 3 to about 50 weight percent of propylene glycol, in water having a pH in the range of about 3.5 to about 6. In a preferred embodiment the composition also includes a polyethylene glycol. In other preferred embodiments the composition can include physiologically tolerable preservatives, such as parabens and chelating agents, and/or electrolyte salts such as sodium chloride.

The gel compositions of the present invention provide a relatively sustained release of the active agent (benzydamine hydrochloride), and can remain in contact with vaginal tissue for a time period sufficient to release substantially all of the active agent. The gel compositions of the present invention are particularly well suited for the treatment of both Gram-positive and Gram-negative bacterial infections of the vagina.

A method aspect of the present invention involves contacting the vagina of a human or veterinary patient suffering from a bacterial vaginal infection with a therapeutically effective amount of a gelled pharmaceutical composition of the present invention.

### DETAILED DESCRIPTION OF PREFERRED EMBODIMENTS

As used herein and in the appended claims, the term “carbomer polymer” and grammatical variations thereof, refers to relatively high molecular weight, acrylic acid polymers crosslinked with allyl sucrose or allylpentaerythritol and which form a gel when dispersed in water at a pH of greater than about 3.5. Carbomer polymers suitable for use in the gelled pharmaceutical compositions of the present invention are commercially available under the trade name CARBOPOL® from Noveon, Inc., Brecksville, Ohio. Carbomer polymers are also described in U.S. Pat. No. 4,808,411 to Lu et al., the relevant disclosure of which is incorporated herein by reference.

As used herein and in the appended claims the term “gel” in reference to aqueous pharmaceutical compositions, means that the composition remains relatively non-fluid at human body temperature (about 37° C.).

The terms “pharmaceutically acceptable”, “physiologically tolerable”, and grammatical variations thereof, as used herein and in the appended claims as they refer to electrolytes (e.g., salts), bases, diluents, preservatives, and other excipients, are used interchangeably and represent that the materials are capable of topical administration to human skin and to the human vagina without the production of undesirable physiological effects such as irritation, itching, stinging, or systemic effects such as nausea, dizziness, and the like.

The term “vagina” as used herein and in the appended claims encompasses the vaginal region generally, including also the vulva and the cervix, of a human or veterinary patient.

The term “therapeutically effective amount” as used herein and in the appended claims, in reference to pharmaceutical compositions, means an amount of pharmaceutical composition that will elicit the biological or medical response of a patient that is sought by a clinician.

A gelled pharmaceutical composition of the present invention is an aqueous gel comprising benzydamine hydrochloride as the active anti-microbial agent. The chemical structure of benzydamine hydrochloride is shown in formula (I).

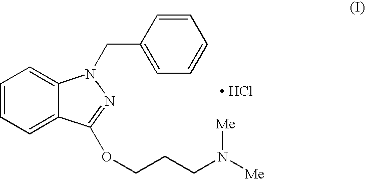

Benzydamine hydrochloride (B-HCl) is a NSAID that also has anti-bacterial activity against both Gram-positive and Gram-negative bacteria. The compositions of the present invention preferably include about 0.1 to about 2 weight percent of B-HCl, more preferably about 0.5 to about 1 weight percent, on a total composition weight basis.

The compositions of the present invention also include a carbomer polymer as a gelling agent and carrier for the active anti-microbial agent. The carbomer polymer is present in the composition preferably in an amount in the range of about 0.5 to about 4 weight percent, more preferably about 1 to about 3 weight percent, on a total composition weight basis. Gelling of the composition is accomplished by neutralizing an aqueous mixture containing the carbomer polymer and B-HCl to a pH preferably in the range of about 3.5 to about 6, more preferably in the range of about 4 to about 5. The mixture is preferably neutralized with a physiologically tolerable base such as an alkali metal hydroxide (e.g., sodium hydroxide and potassium hydroxide), ammonia, or a pharmaceutically acceptable organic amine, such as an alkanolamine (e.g., triethanolamine), an amino acid (e.g., glycine, alanine, lysine, arginine), and the like. Most preferably the neutralization is effected with sodium hydroxide or 6-amino-1-hexanoic acid

The compositions of the present invention also include propylene glycol in an amount in the range of about 3 to about 50 weight percent, on a total composition weight basis. Without being bound by theory, it is thought that the propylene glycol provides improved solubilization of a benzydamine/carbomer salt complex present in the composition.

The compositions of the present invention optionally can include a polyethylene glycol, a physiologically tolerable preservative, and a physiologically tolerable electrolyte, as well as any other pharmaceutically acceptable exipients, so long as the optional components do not interfere with the gelling of the composition or with the release of the benzydamine hydrochloride in the vagina.

The term “polyethylene glycol” as used herein and in the appended claims refers to a homopolymer of ethylene glycol monomers units, also commonly referred to as oxyethylene or ethylene oxide monomer units. Polyethylene glycols (PEG) have a general formula corresponding to HO-(CH₂CH₂O)ₙ—H, where n represents the average number of monomer units in the polymer. When the compositions of the present invention include polyethylene glycol, n is preferably a number in the range of about 5 to about 250 (i.e., the polymer has an average molecular weight in the range of about 200 to about 11,000 Daltons), more preferably n is a number in the range of about 9 to about 180 (i.e., an average molecular weight in the range of about 400 to about 8000 Daltons). The compositions of the present invention also include propylene glycol, preferably in an amount in the range of about 3 to about 50 weight percent, more preferably about 15 to about 50 weight percent, on a total composition weight basis. PEG can be used to increase the solubility of the benzydamine/carbomer salt.

A polyethylene glycol when present in the compositions, preferably is included in an amount in the range of about 25 to about 35 weight percent, on a total composition weight basis.

Suitable physiologically tolerable preservatives include bacterostats, preservatives, inhibitors, and the like, such as methyl, ethyl, propyl, and butyl esters of parahydroxybenzoic acid (paraben); propyl gallate; sorbic acid and its sodium and potassium salts; propionic acid and its calcium and sodium salts; 6-acetoxy-2,4-dimethyl-m-dioxane; 2-bromo-2-nitropropane-1,3-diol; salicylanilides such as dibromosalicylanilide and tribromosalicylamilide, the cis isomer of 1-(3-chloroallyl-3,5,7-triaza-1-azanidadamantane chloride; hexachlorophene; sodium benzoate; chelating agents such as ethylene diaminetetraacetic acid (EDTA), citric acid, and their alkali metal and alkaline earth metal salts; phenolic compounds such as butyl hydroxyanisol, butyl hydroxytoluene, chloro- and bromo-cresols, and chloro- and bromo-oxylenols; quaternary ammonium compounds such as benzalkonium chloride; aromatic alcohols such as 2-phenylethyl alcohol and benzyl alcohol; chlorobutanol; quinoline derivatives such as iodochlorohydroxyquinoline; and the like.

Preferred physiologically tolerable preservatives include parabens (e.g., methyl paraben, propyl paraben, and the like), chelating agents (e.g, EDTA or a physiologically tolerable salt thereof, such as sodium edatate, disodium edatate, and the like), and mixtures thereof. Preferably, the preservative is included in an amount in the range of about 0.05 to about 0.2 weight percent, on a total composition weight basis.

Preferred physiologically tolerable electrolytes include alkali metal chlorides, such as sodium chloride, alkali metal salts of organic acids, such as sodium acetate, and the like. Sodium chloride is particularly preferred. When included in the composition, the electrolyte preferably is present in an amount in the range of about 0.1 to about 1 percent by weight, more preferably about 0.5 to about 0.9 weight percent, on a total composition weight basis.

Pharmaceutically acceptable excipients which can be included in the gelled pharmaceutical compositions of the present invention include, for example, physiologically tolerable surfactants, solvents, buffering agents, colorants, fragrances, and the like, which are well known in the art.

A preferred embodiment of the gelled pharmaceutical composition of the present invention comprises, on a total composition weight basis, about 0.5 to about 1 weight percent of benzydamine hydrochloride, about 1 to about 3 weight percent of a carbomer polymer, and about 3 to about 30 weight percent of propylene glycol, in water having a pH in the range of about 3.5 to about 6. A particularly preferred embodiment of the composition also includes about 25 to about 35 weight percent of a polyethylene glycol.

A method aspect of the present invention is a method of treating vaginal infections comprising contacting the vagina of a patient suffering from a bacterial vaginal infection with a therapeutically effective amount of a gelled pharmaceutical composition of the present invention. Preferably the gelled pharmaceutical composition comprises, on a total composition weight basis, about 0.1 to about 2 weight percent of benzydamine hydrochloride, about 1 to about 4 weight percent of a carbomer polymer, and about 3 to about 50 weight percent of propylene glycol, in water having a pH in the range of about 3.5 to about 6.

A presently preferred technique for contacting the vagina with a gelled pharmaceutical composition of the present invention is to extrude a the gelled composition through a tubular applicator from a storage vessel, such as a syringe, squeezable tube, or the like, into the patient's vagina. The volume of gelled composition so contained within a single such vessel is conveniently and preferably selected so as to constitute a single dose, or two doses, or the like, so as to facilitate administration of a desired controlled dose to the patient's vagina. The storage vessel is initially sealed, but is opened at the time of use. If more than a single dose is present, the vessel is preferably resealable by a suitable closure means. Another presently preferred method of application is a pre-filled unit-dose vaginal applicator.

Another presently preferred technique is to employ a single use packet (such as a small envelope-like structure, or the like) containing an intended single unit dose. The packet is initially sealed, but is opened at the time of use by tearing, cutting, or the like at a desired or planned location in the packet after which the packet is manually squeezed so that the contents are directly administrable as desired

Generally, the dosage of the composition to be contacted with the patient's vagina can vary with the age, condition, and extent of the injury suffered by the patient, and can be determined by one of skill in the art. The dosage can also be adjusted by the individual physician in the event of any complication.

The quantity of benzydamine hydrochloride contained in a unit dose is generally at least about 5 milligrams (mg), and preferably is not more than about 100 mg. A typical and presently more preferred unit dose in a gel vehicle is in the range of about 20 to about 50 mg per dose.

Such a quantity can be administered 1 to 2 times daily (i.e., at spaced intervals during a 24 hour period) in a single day or over a period of up to about 7 days. A typical daily dose thus delivered can range from about 20 to about 100 mg. The usual total dose during the course of therapy for the gelled pharmaceutical compositions of the present invention is in the range of about 60 mg to about 500 mg. A presently preferred administration procedure is to employ a unit dose of about 4 grams of the gel (delivering a dose of about 20 mg of benzydamine hydrochloride) administered once or twice daily for a period of about 3 days, thereby to deliver a total dose in the range of about 60 mg to about 120 mg. Those skilled in the art will appreciate that the foregoing dose levels are provided illustratively, and that higher and lower dose levels can be employed without departing from the spirit and scope of the present invention.

Preferably, the composition will remain in contact with the patient's vagina for a period of time sufficient for substantially all of the active agent (i.e., benzydamine) to be released from the gel. The gelled composition preferably will remain in contact with the patient's vagina for a period of time in the range of about 12 to about 36 hours.

Another aspect of the present invention is an article of manufacture comprising packaging material and a gelled pharmaceutical composition of the invention within the packaging material. The gelled pharmaceutical composition is present in an amount sufficient to treat a bacterial vaginal infection in a patient, preferably in an amount equivalent to at least one unit dose. The packaging material comprises a label that indicates that the gelled pharmaceutical composition can be used for treating bacterial vaginal infections. Preferably the label includes other printed indicia such as a listing of ingredients, the manufacturer's name and address, and the like. Preferably the packaging material also includes a printed insert including detailed information on the composition, its method of administration for treatment of bacterial vaginal infections, side effects, contraindications, and the like indicia, which may be required by governmental agencies responsible for regulation of pharmaceutical products.

The following non-limiting examples further illustrate the present invention.

### EXAMPLE 1

### Preparation of a Gelled Benzydamine Hydrochloride Composition A of the Present Invention

About 2 parts by weight of powdered carbomer polymer (CARBOPOL® 974P from Noveon Inc.) was carefully dispersed in about 58 parts by weight water containing about 0.05 parts by weight of EDTA. About 15 parts by weight of propylene glycol containing about 0.08 parts by weight methyl paraben and about 0.02 parts by weight propyl paraben was added to the carbomer slurry. Benzydamine hydrochloride (about 0.5 parts by weight) was dissolved in water (about 10 parts by weight). The benzydamine solution was added to the carbomer slurry. Finally, the pH was adjusted to about 4.7 with 5% aqueous sodium hydroxide (about 10 parts by weight) and additional water was added to bring the total content of the composition to about 100 parts by weight. The resulting gelled benzydamine composition (Composition A) had a white, creamy appearance.

### EXAMPLE 2

### Preparation of a Gelled Benzydamine Hydrochloride Compositions B-O of the Present Invention

Gelled benzydamine hydrochloride Compositions B-O were prepared following the general procedure of Example 1. The pH and amounts of each of the components in the compositions are provided in Table 1, in percent by weight (pbw).

TABLE 1
Gelled Pharmaceutical Compositions

| | | B-HCl | Carb. | PG | | Other |
|---|---|---|---|---|---|---|
| Comp. | pH | (pbw) | (pbw) | (pbw) | Form | Components |
| A | 4.7 | 0.5 | 2 | 15 | wg | * |
| B | 5.5 | 0.5 | 2 | 15 | wg | * |
| C | 5.4 | 1 | 2 | 15 | wg | * |
| D | 3.9 | 1 | 2 | 48.5 | hg | ** |
| E | 5 | 1 | 1.5 | 48.75 | hg | ** |
| F | 5 | 0.5 | 2 | 15 | cg | PEG-400 (30 pbw)** |
| G | 5.4 | 1 | 2 | 15 | cg | PEG-400 (35 pbw)** |
| H | 4.7 | 0.2 | 2 | 16 | cg | PEG-400 (30 pbw)** |
| I | 4.9 | 0.5 | 2 | 15 | wg | * |
| J | 5.5 | 0.5 | 2 | 15 | wg | NaCl (0.9 pbw) * |
| K | 5.6 | 0.9 | 1.8 | 13.8 | hg | PEG-400 (11.5 pbw) * |
| L | 4.9 | 0.5 | 2.5 | 15 | hg | PEG-400 (25 pbw) * |
| M | na | 1 | 2.5 | 15 | wg | * |
| N | 5 | 1 | 3 | 3 | wg | * |
| O | 5.5 | 1 | 3 | 3 | wg | NaCl (0.9 pbw) * |

* 0.05 percent by weight EDTA, 0.08 percent by weight methyl paraben, 0.02 percent by weight propyl paraben, and a quantity of water sufficient to make up 100 percent by weight.
**a quantity of water sufficient to make up 100 percent by weight).
PEG-400 refers to a polyethylene glycol having a weight average molecular weight of about 400 (i.e., having about 9 oxyethylene monomer units).

In Table 1, “Form” refers to the appearance of the gel; cg=a clear, transparent appearance, hg=a hazy appearance, wg=a white, creamy appearance; “pbw” refers to percent by weight; “Carb.” refers to CARBOPOL® 974 carbomer polymer; and “PG” is propylene glycol. The pH values in Table 1 have been rounded to the nearest tenth of a pH unit. The pH of each formulation was adjusted to the indicated value by addition of a sufficient quantity of 5% aqueous sodium hydroxide.

### EXAMPLE 3

### Evaluation of Antibacterial Activity of Compositions A-J of the Present Invention

About 1 grams of each gelled composition was deposited on an agar plate that was previously inoculated with E. coli (ATCC #11229), a Gram-negative bacterium, and the plates were incubated for about 20 to about 28 hours at a temperature of about 35 to about 39° C. Antibacterial activity was evaluated by measuring the diameter of the zone of inhibition (i.e., a clear zone with no bacterial colonies present, measured in millimeters) around the deposited gel.

Similar plates were prepared wherein the inoculant was Staphylococcus aureus (ATCC #6538), a Gram-positive bacterium.

A commercially available vaginal cream formulation of benzydamine hydrochloride, TANTUM ROSA® cream, available from Angelini Pharmaceuticals, Rome, Italy was also evaluated, for comparison. According to the manufacturer's product literature TANTUM ROSA® cream contains about 0.5% benzydamine hydrochloride in a cream base containing propylene glycol, saturated triglycerides, ceteth 20, hydroxyethyl cellulose, sodium citrate dihydrate, citric acid monohydrate, benzoic acid, and purified water. In addition, a simple aqueous solution containing 0.5% by weight of benzydamine hydrochloride in deionized water was tested as a positive control. All materials were tested in triplicate.

The results of the inhibition tests are provided in Table 2. As is apparent from the data in Table 2, the gelled benzydamine compositions of the present invention are active against both Gram-negative and Gram-positive bacteria. The simple aqueous solution of benzydamine hydrochloride was effective against both species of bacteria. These results indicate that the gelled compositions of the present invention provide a treatment for bacterial vaginal infections that is superior to the commercial benzydamine cream product, and in an aqueous gelled form suitable for adhesion to vaginal tissue for prolonged exposure to the active agent.

TABLE 2
Bacterial Inhibition

| | E. coli | Staphylococcus |
|---|---|---|
| Comp. | clear zone, mm | clear zone, mm |
| TANTUM ® | Not Tested | 0 |
| B-HCl | 12 | 14 |
| A | 19 | 21 |
| B | 17 | 18 |
| C | 18 | 23 |
| D | 12 | 7 |
| E | 16 | 15 |
| F | 22 | na |
| G | 25 | na |
| H | 15 | na |
| I | 16 | na |
| J | 23 | na |

### EXAMPLE 4

### Evaluation of Benzydamine Release Rates from Compositions of the Present Invention into a Saline Medium

About 0.18 grams of Composition C from Example 2 was placed in a section (about 6 cm) of cellulose dialysis tubing having a molecular weight cut-off of about 12,000 Daltons (average flat width of about 9 mm, Sigma Chemical Company, St. Louis, Mo.) and the ends of the tubing were sealed with plastic clips. The sealed tube was suspended horizontally in a petri dish containing about 50 mL of 0.9% by weight aqueous sodium chloride (i.e., physiological saline medium). The saline medium was continually stirred and the amount of released benzydamine hydrochloride in the medium was periodically determined by measuring the UV absorbance of the medium at a wavelength of about 308 nm with a spectrophotometer and comparing the measured absorbance with an appropriate calibration curve, as is well known in the art. The medium was replaced with fresh saline medium after each determination of released benzydamine hydrochloride to prevent equilibration and maintain a sink for the benzydamine hydrochloride. Determinations of released benzydamine hydrochloride were performed about 1.5 hours, 4 hours, 8, hours, 12 hours, and 24 hours after initial emersion of the dialysis tubing containing the gel in the saline medium.

This procedure was repeated with Composition E (about 0.2 grams of gel), as well as for TANTUM ROSA® cream (about 0.12 grams). The release data are provided in Table 3.

TABLE 3
Benzydamine Release Rate in Saline.

| Comp. | 1.5 hr | 4 hr | 8hr | 12 hr | 24hr |
|---|---|---|---|---|---|
| C | 15% | 40% | 69% | 88% | 100% |
| E | 15% | 40% | 77% | 97% | 97% |
| TANTUM ® | 69% | 100% | 100% | 100% | 100% |

### EXAMPLE 5

### Evaluation of Benzydamine Release Rates from Compositions of the Present Invention into Water and Saline Media

A modification of the procedure of Example 4 was followed with Compositions C and E, except that the dialysis medium was deionized water during the first 24 hours. After about 24 hours, the water medium was replaced by physiological saline and the release of benzydamine hydrochloride was followed for an additional 24 hours. The results are provided in Table 4.

TABLE 4
Benzydamine Release Rate in Water and Saline.

| Water | 2.5 hr | 4 hr | 8 hr | 12 hr | 24 hr |
|---|---|---|---|---|---|
| C | 4% | 5% | 5% | 5% | 7% |
| E | 5% | 5% | 5% | 6% | 8% |
| Saline | 26 hr | 28 hr | 32 hr | 36 hr | 48 hr |
| C | 33% | 58% | 85% | 100% | 100% |
| E | 22% | 38% | 64% | 82% | 100% |

The data in Tables 3 and 4 indicate that the gelled pharmaceutical compositions of the present invention provide a relatively sustained release of benzydamine hydrochloride into a saline medium in comparison with a commercial benzydamine hydrochloride cream formulation. Not intending to be bound by theory, the significantly slower benzydamine hydrochloride release rate in water medium compared to saline medium suggests that the benzydamine hydrochloride is released by an ion exchange mechanism (i.e., sodium ion displaces the positively-charged protonated form of benzydamine from the insoluble carbomer polymer releasing benzydamine hydrochloride into the water phase). This further suggests that the benzydamine is present in the compositions in the form of a salt with the carbomer polymer (i.e., a new composition of matter), rather than merely being dissolved in the aqueous medium contained in the swollen carbomer gel.

The gelled pharmaceutical compositions of the present invention provide a convenient and effective treatment for bacterial vaginal infections. The gelled formulation affords a mucoadhesive vehicle for delivery of benzydamine hydrochloride, which can remain in contact with vaginal tissue for a prolonged period of time. The gelled pharmaceutical compositions of the present invention also provide a sustained release dosage form of benzydamine hydrochloride that is effective against both Gram-positive and Gram-negative bacteria.

The foregoing specification enables one skilled in the art to practice the invention. Indeed, various modifications of the invention in addition to those shown and described herein will become apparent to those skilled in the art from the foregoing description and fall within the scope of the appended claims.

We claim:

1. A gelled pharmaceutical composition that comprises, on a total composition weight basis, about 0.1 to about 2 weight percent of benzydamine hydrochloride, about 0.5 to about 4 weight percent of a carbomer polymer, and about 3 to about 50 weight percent of propylene glycol, in water having a pH in the range of about 3.5 to about 6.

2. The composition in accordance with claim 1 wherein the benzydamine hydrochloride is present in the composition in an amount in the range of about 0.5 to about 1 weight percent on a total composition weight basis.

3. The composition in accordance with claim 1 further comprising polyethylene glycol.

4. The composition in accordance with claim 3 wherein the polyethylene glycol is present in the composition in an amount in the range of about 25 to about 35 weight percent on a total composition weight basis.

5. The composition in accordance with claim 3 wherein the polyethylene glycol comprises about 5 to about 250 ethylene glycol monomer units.

6. The composition in accordance with claim 1 further comprising at least one physiologically tolerable preservative agent.

7. The composition in accordance with claim 6 wherein the preservative agent is a paraben, a chelating agent, or a mixture thereof.

8. The composition in accordance with claim 7 wherein the paraben is methyl paraben, propyl paraben, or a mixture thereof.

9. The composition in accordance with claim 7 wherein the chelating agent is EDTA or a salt thereof.

10. The composition in accordance with claim 1 wherein the composition has a pH in the range of about 4 to about 6.

11. The composition in accordance with claim 1 further comprising about 0.1 to about 1 weight percent of a physiologically tolerable electrolyte salt.

12. The composition in accordance with claim 11 wherein the electrolyte salt is sodium chloride.

13. The composition in accordance with claim 12 wherein the sodium chloride is present in an amount in the range of about 0.5 to about 0.9 percent by weight on a total composition weight basis.

14. A gelled pharmaceutical composition that comprises, on a total composition weight basis, about 0.5 to about 1 weight percent of benzydamine hydrochloride, about 1 to about 3 weight percent of a carbomer polymer, and about 15 to about 50 weight percent of propylene glycol, in water having a pH in the range of about 3.5 to about 6.

15. The composition in accordance with claim 14 further comprising polyethylene glycol.

16. The composition in accordance with claim 15 wherein the polyethylene glycol is present in the composition in an amount in the range of about 25 to about 35 weight percent on a total composition weight basis.

17. The composition in accordance with claim 15 wherein the polyethylene glycol comprises about 5 to about 250 ethylene glycol monomer units.

18. The composition in accordance with claim 14 further comprising at least one physiologically tolerable preservative agent.

19. The composition in accordance with claim 18 wherein the preservative agent is a paraben, a chelating agent, or a mixture thereof.

20. The composition in accordance with claim 19 wherein the paraben is methyl paraben, propyl paraben, or a mixture thereof.

21. The composition in accordance with claim 19 wherein the chelating agent is EDTA or a salt thereof.

22. The composition in accordance with claim 14 wherein the composition has a pH in the range of about 4 to about 6.

23. The composition in accordance with claim 14 further comprising about 0.5 to about 0.9 weight percent of sodium chloride.

24. An article of manufacture comprising packaging material and a gelled pharmaceutical composition within the packaging material; the gelled pharmaceutical composition being present in an amount sufficient to treat a bacterial vaginal infection in a patient; the packaging material comprising a label that indicates that the gelled pharmaceutical composition can be used for treating a bacterial vaginal infection; and the gelled composition comprising, on a total composition weight basis, about 0.1 to about 2 weight percent of benzydamine hydrochloride, about 0.5 to about 4 weight percent of a carbomer polymer, and about 3 to about 50 weight percent of propylene glycol, in water having a pH in the range of about 3.5 to about 6.

25. The article of manufacture in accordance with claim 24 wherein the packaging material further includes an insert comprising printed indicia selected from the group consisting of a description of the ingredients in the composition, the method of administration of the composition for treatment of bacterial vaginal infections, the side affects of the composition, contraindications to the use of the composition, and combinations thereof.

26. The article of manufacture in accordance with claim 24 wherein the gelled pharmaceutical composition further comprises about 25 to about 35 weight percent of a polyethylene glycol, on a total composition weight basis.
